(19) United States
(12) Patent Application Publication
(10) Pub. No.: US 2003/0036103 A1
(43) Pub. Date: Feb. 20, 2003

(54) Methods and compositions for diagnosis and treatment of vascular conditions

(76) Inventors: Sivaram Pillarisetti, Norcross, GA (US); Dongyan Wang, Norcross, GA (US); Uday Saxena, Atlanta, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/210,896

(22) Filed: Jul. 31, 2002

Related U.S. Application Data
(60) Provisional application No. 60/309,012, filed on Jul. 31, 2001.

(51) Int. Cl. A61K 31/727; G01N 33/574; G01N 33/53

(52) U.S. Cl. 435/7.23; 435/7.5; 514/56

(57) ABSTRACT

The present invention is directed to methods and compositions for the diagnosis and treatment of vascular conditions, particularly diabetes and atherosclerosis. The present invention comprises methods and compositions for determining the expression or activity of enzymes effecting HSPG, preferably, heparanase. The invention also comprises methods and compositions for treatment of vasculophathic diseases comprising administration of therapeutic compounds that are effective in inhibiting the expression or activity of heparanase.

### RELATED APPLICATIONS

This application claims benefit of U.S. Provisional Application No. 60/309,012 filed Jul. 31, 2001.

### FIELD OF THE INVENTION

The present invention relates to methods and compositions for treatment of vascular conditions, particularly diabetes and atherosclerosis. The present invention is directed to methods and compositions for determining the expression or activity of enzymes affecting heparan sulfate proteoglycan and the use of therapeutic compounds that effect the expression or activity of these enzymes, particularly heparanase.

### BACKGROUND OF THE INVENTION

There are many disease states in humans and animals that are related to changes in vascular conditions. Two of these pathological states, diabetes and its attendant complications and cardiovascular disease, effect a large number of individuals. One of the common characteristics that these disease states share is changes in the vascular condition, particularly increased vascular permeability. The reasons for increased vascular permeability in diabetes and cardiovascular diseases such as atherosclerosis and the resulting albuminuria are not clear.

Changes in urinary albumin levels are seen in diabetics with nephropathy. Diabetic nephropathy develops in 30-40% of individuals with Type I diabetes and 10-40% of those with Type II diabetes. The cause of diabetic nephropathy is still unknown. Albuminuria is also a predictor of ischemic heart disease and generalized vascular disease.

Changes in vascular permeability are related to changes in the basement membrane. The basement membrane is a complex network of fibronectin, laminin, collagen and vitronectin, each of which interact with heparan sulfate side chains of heparan sulfate proteoglycan (HSPG) embedded within the matrix. The basement membrane separates cells and cell sheets from connective tissue and also functions as a highly selective filter. The basement membrane determines cell polarity and cellular metabolism, organizes the proteins in adjacent plasma membranes, induces cell differentiation and plays a role in cell migration.

Heparan sulfate (HS) chains in healthy tissue generally consist of clusters of sulfated disaccharide units separated by minimally sulfated or non-sulfated regions. In diabetes, there is a loss of normally sulfated heparan sulfate in extracellular matrix plasma membranes Changes in HSPG are also seen in atherosclerosis. The mechanism by which tissue HSPG is lost in diabetes and in atherosclerosis is not known.

Heparanase is a mammalian endoglucuronidase that degrades heparan sulfate chains of HSPG. Heparanase has been isolated and characterized from several mammalian cells and has been cloned from human placenta. The expression of this heparanase is induced in metastasizing tumors and has been shown to play a role in the extent of tumor metastasis, permitting the tumor to successfully penetrate endothelial basement membranes.

What is needed are methods and compositions for diagnosing the beginning stages of vascular conditions, and particularly those associated with HSPG changes. What is also needed are therapeutic agents that effect the HSPG concentration changes associated with changes in vascular conditions.

### SUMMARY OF THE INVENTION

The present invention is directed to compositions and methods for the diagnosis and treatment of vasculopathy and vascular conditions. Such methods comprise diagnosis of vascular changes by detecting changes in heparan sulfate proteoglycan (HSPG). More particularly, the present invention comprises methods and compositions for determining the activity of enzymes affecting HSPG and also comprises methods and compositions for altering the activity of such enzymes.

The present invention further comprises methods for detecting the decrease in HSPG and for detecting an increase in albuminuria comprising determining the activity of enzymes which degrade HSPG, preferably enzymes such as heparan sulfate degrading heparanase. In particular, methods comprise detecting the up-regulation of these enzymes. Methods for determining changes in HSPG concentration, vascular changes and increased urinary albumin excretion are used in methods of diagnosis of vascular conditions which are associated with many diseases, including, but not limited to, kidney disease, ischemic heart disease, cardiovascular disease, generalized vascular disease, proliferative retinopathy, and macroangeopathy. Compositions that effect the concentrations of HSPG are used in methods of treatment of such vascular and systemic diseases.

The present invention also comprises methods and compositions for the inhibition of enzymes which effect HSPG levels, amount or activity. Methods and compositions comprising therapeutic agents that block the activity of heparanase or other HSPG degrading enzymes are useful for the treatment of conditions such as diabetic vasculopathy and cardiovascular disease. The present invention also comprises methods and compositions that alter the activity of enzymes which effect HSPG levels.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 is a graph showing the induction of heparanase activity in endothelial cells.

FIGS. 2A, B, and C are photographs showing heparanase activity in mouse cells.

FIG. 3 is a western blot showing heparanase activity in endothelial cells.

FIGS. 4A and C are western blots of endothelial cell secretion of heparanase induced by pro-inflammatory cytokines and inhibited by anti-inflammatory agents.

FIGS. 4B and D are graphs of the changes of heparanase expression in the endothelial cells in 4A and C.

FIG. 5A is a western blot of heparanse expression in endothelial cells treated with TNF α and P13 inhibitors.

FIG. 5B is a western blot of heparanse expression in endothelial cells treated with TNF α and NFκB inhibitors.

FIG. 5C is a western blot of heparanase expression in endothelial cells treated with TNF α and MAP kinase inhibitors.

FIG. 5D is a western blot of heparanse expression in endothelial cells treated with TNF α and caspase inhibitor III.

FIG. 6A is a photograph of aortic tissue section from a three month old wild type mouse which was immunostained for heparanase expression.

FIG. 6B is a photograph of aortic tissue sections of a three month old apoE-null mouse which was immunostained for heparanase expression.

FIG. 6C is a photograph of aortic tissue sections from a one year old apoE null mouse which was immunostained for heparanase expression.

### DETAILED DESCRIPTION OF THE INVENTION

The present invention is directed to methods and compositions for the diagnosis and treatment of pathological changes in vascular tissues. Such changes signal early stages of diseases such as diabetes and atherosclerosis. Methods and compositions for effecting the changes in vascular tissues are disclosed and for effective therapeutic treatments of such diseases.

An increase in albumin level, with its accompanying albuminuria, is a predictor of both ishemic heart disease and generalized vascular disease and shows symptoms such as increased microvascular permeability, increased plasma levels of von Willebrand factor and thrombomodulin and reduced fibrinolytic capacity. (4,6) Microalbuminuria has also been associated with proliferative retinopathy, diabetic nephropathy and macroangeopathy. The causes of increased vascular permeability and the resulting albuminuria are not clear. Though not wishing to be bound by any particular theory, it is believed that loss of anionic charged proteoglycans affecting the structure of basement membranes, is responsible for initial microalbuminuria.

Heparan sulfate proteoglycan (HSPG) is the main glycosaminoglycan component of basement membranes of kidney glomeruli, aortic myomedial cells, mesangium and endothelial plasma membranes. Within the basement membrane of the kidney, it is believed that HSPG not only inhibits the glomerular filtration of albumin, but also contributes to the pore size of the glomeruli. In general, it is believed that HSPG binds lipoprotein lipase, inhibits smooth muscle cell proliferation and has anti-thrombogenic properties.

The basement membrane of tissue consists predominantly of a complex network of adhesion proteins, fibronectin, laminin, collagen and vitronectin. Each of these adhesion proteins interacts with sulfate side chains of HSPG within the matrix. Thus, it is believed that HSPG is a contributor to the integrity of the basement membrane and barrier function. Kidney glomerular basement membrane heparan sulfate may also act to maintain the structural integrity of the glomerular filter and the pore structures that determine size selectivity. Loss of heparan sulfate has been shown to result in loss of anionic charge and albuminuria. Though not wishing to be bound by any particular theory, it is thought that cleavage of HSPG may assist in the disassembly of the extracellular matrix and thereby facilitate cell migration. Although heparanase activity has been described in metastasizing tumors and has been postulated to contribute to cancer metastasis, its role in other disease processes is unclear.

Heparin sulfate is a strong inhibitor of mesangial cell growth, and reduced content of heparan sulfate in the basement membrane has been demonstrated in diabetic patients with mesangial cell expansion and clinical nephropathy. (13-14) There is also a negative correlation between the number of anionic sites representing HSPG in the kidney glomerular basement membrane and urinary albumin secretion.

The concentration of HSPG also negatively correlates with atherosclerosis. (15-16) Increasing amounts of cholesterol in plaques is thought to be related to the concentration of HSPG, which is decreased in aortic tissue. This negative correlation was observed both in normal and atherosclerotic vessels with four- to five-fold more cholesterol found in vessels that have a 50% reduction in HSPG content.

The present invention is directed to detection and control of the effects of molecules such as advanced glycation end products (AGE) and oxidative stress molecules which are involved in the pathogenesis of vascular diseases, such as diabetic vasculopathy, which are believed to induce heparanase activity in endothelial cells. For example, molecules related to hyperglycemia such as AGE, and oxidative stress are agents in the development of diabetic vascular complications. These agents also induce permeability changes in cultured cells.

Nonenzymatic glycation of proteins and AGE are a part of a mechanism by which hyperglycemia leads to diabetic renal disease. Recent research has shown that Amadori-modified albumin, the principal glycated protein in plasma, elicits pathobiologic effects in cultured renal cells that are identical to the physiological changes seen in patients with high ambient glucose. When added to the incubation media of glomerular mesangial and endothelial cells, glycated albumin stimulates protein kinase C activity, increases transforming growth factor-beta (TGF-beta) bioactivity, and induces gene overexpression and enhanced production of extracellular matrix proteins. Glycated proteins alter the permeability properties of the glomerular capillary wall and are preferentially transported across the glomerular filtration barrier and into the mesangial space. The present invention found that AGE induces heparanase in endothelial cells, and while not wanting to be bound to any particular theory, it is theorized that this shows that heparanase mediates the vasculopathic and atherogenic effects of AGE.

In vivo studies also show a role for glycated proteins in the pathogenesis of diabetic nephropathy. Reduction or neutralization of glycated albumin in the db/db mouse model of type 2 diabetes significantly ameliorates the proteinuria, renal insufficiency, mesangial expansion, and overexpression of matrix proteins. In human type 1 diabetes, the plasma-glycated albumin concentration is independently associated with the presence of nephropathy.

Methods of the present invention also comprise abrogating the biologic effects of increased inflammatory cytokines for therapeutic treatments in the management of renal complications in diabetes. An inflammatory cytokine that can contribute to both general inflammation and diabetic vasculopathy is tumor necrosis factor alpha (TNF-α). TNF-α has been implicated in the pathophysiology in a number of acute disease states and can contribute to cell death, apoptosis, and organ dysfunction. Recent evidence also implicates TNF-α as a factor in obesity-associated insulin resistance and the pathogenesis of type 2 diabetes. In addition, it is also believed that TNF-α together with another inflammatory cytokine IL-1β, contributes to the pathogenesis of arthritis. The novel finding of the present invention that TNFα induces heparanase in endothelial cells is theorized to show that heparanase mediates the vasculopathic and atherogenic effects of TNFα.

The present invention also comprises diagnosis of atherosclerosis using methods for detecting heparanase activity and expression. Data presented herein show that lysolecithin, a component of OxLDL (oxidized low-density lipoprotein) induces heparanase expression in endothelial cells. Although hypercholesterolemia is a major risk for atherogenesis, it is theorized that oxidative modification of the major cholesterol-carrying lipoprotein, low-density lipoprotein (LDL), renders it more atherogenic. Not only does OxLDL contribute directly to foam cell formation, it may also adversely affect many other aspects of arterial wall metabolism and thus contribute further to the atherogenic process. OxLDL can induce endothelial dysfunction and permeability changes in vitro. Several of the pathological effects of OxLDL are mediated by its lipid component lysolecithin. The novel finding of the present invention that lysolecithin induces heparanase in endothelial cells shows that heparanase mediates the atherogenic effects of OxLDL and lysolecithin.

The induction of heparanase in mouse models of kidney disease further shows its role in kidney dysfunction. Compared to the kidneys of wild type mice, kidneys from apoE-null mice and db/db mice have high levels of heparanase expression. Both of these types of mice have reduced kidney HSPG. Though not wishing to be bound by any particular theory, it is theorized that because HSPG serves to block the passage of anionic macromolecules through the basement membrane, decreased levels of HSPG account for the increased porosity of basement membrane. Although reduced HS is a common feature in diabetes and atherosclerosis, the reason for this decrease was not known prior to the present invention. In studies that compared HSPG core protein and HS chains in human kidney disease using specific antibodies, the major alteration was found to be a segmental or total absence of staining with anti-HS antibody, which was most pronounced in lupus nephritis, membranous glomerulonephritis and diabetic nephropathy, whereas the HSPG-core staining was unaltered.

The present invention comprises methods and compositions for determining the presence of glycosaminoglycan degrading enzyme activity, particularly heparanase expression, for diagnosis of the presence of vasculopathy. In particular, the methods and compositions of the present invention can also be used to provide treatments for such vasculopathy by administering therapeutic compounds or agents that alter the expression and activity of heparanase and functionally equivalent enzymes having the same relationships with vasculopathy.

An embodiment of the methods of the present invention for diagnosis of early stages of vasculopathy associated with such diseases as diabetes and atherogenic diseases comprises determination of the presence of expression or activity of glycosaminoglycan degrading enzymes, particularly HSPG degrading enzymes. In particular, the methods comprise detection of enzymes, including but not limited to heparanase and other proteoglycan degrading enzymes, and determination of their activity level, using the assays described herein and other immunological and molecular biological techniques. The methods include detection of the expression or activity of proteoglycan degrading enzymes by using the assays described herein as well as other immunological and molecular biological techniques. Tissue and fluid samples from humans or animals suspected of having vasculopathy diseases are assayed to measure the presence of nucleic acids associated with enzymes such as heparanase. Samples can also be tested by immunoassays for enzyme nucleic acids or proteins. Such assays are known to those skilled in the art and include, but are not limited to, assays such as PCR, RT-PCR, Northern and Southern blots, automated assays, and other assays using specific probes for enzymes, preferably heparanase nucleic acids. Biological assays include, but are not limited to, assays which determine the presence of RNA, preferably mRNA, encoding for heparanase. Immunoassays include, but are not limited to, assays which use specific antibodies for enzymes capable of affecting HSPG, preferably heparanase, or antibodies specific for nucleic acids encoding enzymes capable of affecting HSPG, preferably heparanase. Such immunoassays are known in the art and include ELISA, Western blots, and in situ immunohistological staining. The presence of expression or activity of enzymes capable of affecting HSPG, preferably heparanase, provides a diagnosis of vasculopathy. Diagnosis may also be based upon other tests for determining the presence of disease. Determination of the presence of expression or activity of these enzymes, preferably heparanase, can also be used to monitor the effects of thereapeutic regimens or other treatment activities once the disease diagnosis has been made.

The present invention also comprises methods for determining and monitoring the effects of administering compositions comprising therapeutic agents that alter the activity of enzymes associated with vasculopathic changes. Preferred methods comprise administration of compositions that inhibit the activity of enzymes associated with vasculopathic changes. Compositions comprising therapeutic agents that inhibit the activity of enzymes, preferably heparanase or other proteoglycan degrading enzymes, are used to treat such vasculopathy. Assays for inhibiting heparanase are used as screening assays to determine such therapeutic agents. Such assays are disclosed herein, and changes in heparanase activity or expression can also be assayed by other methods known in the art.

A preferred method of the present invention comprises a composition comprising biotin-HS that is mixed with a sample, such as a tumor sample, bodily fluid, or other fluid suspected of having proteoglycan degrading enzyme activity such as heparinase activity, to form a reaction mixture. This sample may be pretreated to remove contaminating or reactive substances such as endogenous biotin. After incubation, an aliquot or portion of the reaction mixture is removed and placed in a biotin-binding plate. After washing with buffers, a Streptavidin-enzyme conjugate is added to the biotin-binding plate. Reagents for the enzyme are added to form a detectable color product. For example, a decrease in color formation, from a known standard, indicates there was heparinase activity in the sample. The biotin-binding plate comprises any means for binding biotin, preferably to a solid surface.

The present invention can also be used to establish a normative standard of enzyme activity by using the assays of the present invention to determine normal levels of enzyme activity in a population. This standard can then be used as a comparison for enzyme activity, particularly heparinase activity in an individual wherein an increase in enzyme activity from the standard would diagnose or predict vasculopathy.

In general, a preferred method comprises attaching one member of a binding partner (first binding partner) to a substrate for the enzyme to be measured forming the substrate-binding partner. Incubation with a biological sample potentially comprising the enzyme to be measured creates a reaction mixture. The biological sample can be any bodily fluid including, but not limited to blood, serum, saliva, tissue fluid, urine, tears or plasma, tissue, including cells, a biopsy section, a tumor, or neoplasm sample. A portion or the whole reaction mixture, depending on the amount needed, is then mixed with a first complementary binding partner, so that the substrate-binding partner and the first complementary binding partner are bound together. This is the first binding reaction. After incubating to allow for binding, washings are performed. A second complementary binding partner, complementary to the first binding partner which is attached to the substrate, is added. This second complementary binding partner may or may not be the same as the first complementary binding partner. This is the second binding reaction. The second complementary binding partner in the second binding reaction is labeled in a manner that is detectable. For example, the second complementary binding partner is labeled with an enzyme that causes a detectable color change when the appropriate reaction conditions exist.

Preferred methods comprise use of binding partners, including but not limited to, biotin and Streptavidin. “Complementary binding partner” means one of the pair of the binding partners, such as biotin and Streptavidin or an antibody and its antigen. The biotin is the complementary binding partner of Streptavidin, Streptavidin is the complementary binding partner of biotin. An antibody that specifically binds biotin is also a complementary binding partner of biotin.

The enzyme of the sample, for which the activity or presence is being detected, can be any of the enzymes that are involved in vascular changes, including but not limited to, any enzymes with proteoglycan-degrading activity, chondroitinase, heparan sulfate endoglycosidase, heparan sulfate exoglycosidase, polysaccharide lyases, keratanase, hyaluronidase, glucanase, amylase, and other glycosidases and enzymes, herein referred to as “proteoglycan degrading enzyme.”

The labeled second complementary binding partner, in the above method, the enzyme labeled-streptavidin, can be labeled with any detectable label, including but not limited to, enzymes, dyes, chemiluminescence, and other methods known in the art. A preferred method comprises labeling with an enzyme that produces a detectable color change when its substrate is present. This method is safe, easy, effective and can be used in both qualitative and quantitative methods.

Using the above methods, the amount of enzyme activity in a sample can be determined. Also, the above methods can be used to determine compounds that can alter, inhibit or stimulate enzyme activity. For example, a composition comprising the compound of interest is added to a known amount of heparinase either before or during the incubation of the heparinase and its substrate-binding partner. Following the other steps of the assay results in a measured amount of label indicating enzyme activity labels. If the compound alters the activity of the heparinase, the assay methods of the present invention will show a change in the amount of detected label. Such assays are used for high throughput determination of the activity of compounds.

Similar assays can be used to determine compounds that alter, inhibit or stimulate enzyme activity where the enzyme activity has been up-regulated. For example, a composition comprising the compound of interest is added to a sample comprising cells that have been treated with enzyme inducing compounds as determined by the present invention such as AGE, TNFα, or OxLDL. If the compound alters the activity of the up-regulated enzyme, the assay methods of the present invention will show a change in the amount of detected label. Such assays are used for high throughput determination of the activity of compounds and can be used to further isolate useful compounds.

The compositions and methods of the present invention can be used to diagnose the presence of metastases or the metastatic potential of tumors, which includes cancer, neoplastic growth, either initial or return metastatic growth. A preferred embodiment of the present invention comprises the following methods. Patients suspected of having one or several tumors, either in an initial finding or in a return of tumor growth, provide a biological sample for testing. The biological sample may be pretreated to remove endogenous biotin. The sample is used in the assays of the present invention. An increase in the proteoglycan degrading enzyme activity, particularly heparanase activity, or a high level of proteoglycan degrading enzyme activity, is indicative of tumor or metastases presence. Other tests known to those skilled in the art can also be used in combination with the assays of the present invention.

Another use of the present invention is for determining compounds that influence the proteoglycan degrading activity in cells, tissues or whole body responses. Because the present invention comprises assays for quantitatively measuring proteoglycan degrading activity, compounds that inhibit or enhance that activity can be determined easily using such assays. For example, once a known amount of heparanase activity is determined from the assays of the present invention, compounds can be added to the assay and the amount of inhibition can be determined. These compounds can be, but are not limited to, small organic molecules, peptides, peptoids, or polynucleotides that alter the enzymatic activity or decrease the biological stability of the enzyme. The present invention comprises high throughput assays which can measure the effects on enzyme activity levels by many different compounds. For example, the effect of compounds on the inhibition of proteoglycan degrading activity can be measured in vitro or in vivo, using any type of sample known to those skilled in the art.

Compositions comprising therapeutic agents that are effective in inhibiting enzyme activity or expression, preferably that of heparanase, are administered to animals having or suspected of having vasculopathy. These agents may be in administered in doses ranging from 10 ng to 10 g, preferably 10 ng to 5 g, more preferably 10 ng to 1 g, preferably 5 ng to 5 g, still more preferably 5 ng to 0.5 g, preferably 5 ng to 0.05 g, more preferably 1 ng to 0.5 g, preferably 5 ng to 0.005 g, still more preferably 5 ng to 0.0005 g, most preferably a dosage which generates a serum level of 5 ng to 10 ng. Effective amounts of such compositions are administered to animals in dosages that are safe and effective. Routes of administration include intravenous, subcutaneous, transdermal, nasal, inhalation, and other routes that are known to those in the art. Such therapeutic agents may be used in conjunction with other therapeutic agents or altered patient activities, such as changes in exercise or diet.

It must be noted that, as used in this specification and the appended claims, the singular forms “a,” “an,” and “the” include plural referents unless the context clearly dictates otherwise.

The term “treating” “treatment” “treat” as used herein includes preventative, emergency, and long-term treatment.

The terms “drug”, “agent”, “therapeutic agent”, “medication”, and the like are considered to be synonymous and all refer to the component that has a physiological effect on the individual to whom the composition is administered.

As used herein, “effective amount” means an amount of a composition comprising a therapeutic agent that is sufficient to provide a selected effect and performance at a reasonable benefit/risk ratio attending any medical treatment.

The compositions of the present invention may further include pharmaceutically acceptable carriers. The compositions may also include other medicinal agents, pharmaceutical agents, carriers, adjuvants, diluents and other pharmaceutical preparations known to those skilled in the art. Such agents are generally described as being biologically inactive and can be administered to patients without causing deleterious interactions with the therapeutic agent. Examples of carriers or excipients for oral administration include corn starch, lactose, magnesium stearate, microcrystalline cellulose and stearic acid, povidone, dibasic calcium phosphate and sodium starch glycolate. Any carrier suitable for the desired administration route is contemplated by the present invention.

The therapeutic agents of the present invention may be administered in effective amounts in pharmaceutical formulations comprising admixtures with suitable pharmaceutical diluents, excipients or carriers. The formulations may be in tablets, capsules, elixirs or syrups. Additionally, the formulations of the compositions of the present invention may comprise sustained release formulations that provide rate controlled release of any one or more of the therapeutic agents. Sustained release formulations are well known in the art.

Administration of the therapeutic agents of the present invention is dependent on the route of administration and the formulation of the compositions, for example, whether the formulation is designed for quick release or long term release. The doses provided herein may be amended by those skilled in the art, such as physicians or formulation pharmacists. Doses may differ for adults from those for pediatric patients.

The routes of administration for agents is chosen according to the speed of absorption desired and the site of action of the agent. Various routes of administration of the present invention are presented herein.

The formulations include those suitable for oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration. The formulations may conveniently be presented in unit dosage form and may be prepared by conventional pharmaceutical techniques. Such techniques include the step of bringing into association the therapeutic agent and the pharmaceutical carrier(s) or excipient(s). In general, the formulations are prepared by uniformly and intimately bringing into association the therapeutic agent with liquid carriers or finely divided solid carriers or both, and then, if necessary, shaping the product.

Formulations of the present invention suitable for oral administration may be presented as discrete units such as capsules, cachets or tablets each containing a predetermined amount of the therapeutic agent; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the therapeutic agent in a free-flowing form such as a powder or granules, optionally mixed with a binder, lubricant, inert diluent, preservative, surface therapeutic or dispersing agent. Molded tablets may be made by molding, in a suitable machine, a mixture of the powdered compound moistened with an inert liquid diluent. The tablets may be optionally coated or scored and may be formulated so as to provide a slow or controlled release of the therapeutic agent therein.

Formulations suitable for topical administration in the mouth include lozenges comprising the ingredients in a flavored basis, usually sucrose and acacia or tragacanth; pastilles comprising the therapeutic ingredient in an inert basis such as gelatin and glycerin, or sucrose and acacia; and mouthwashes comprising the therapeutic agent to be administered in a suitable liquid carrier.

Formulations suitable for topical administration to the skin may be presented as ointments, creams, gels and pastes comprising the therapeutic agent to be administered in a pharmaceutically acceptable carrier. A preferred topical delivery system is a transdermal patch containing the therapeutic agent to be administered.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose or through devices designed to deliver a powdered formulation to the nose or lungs. Suitable formulations, wherein the carrier is a liquid, for administration, as for example, a nasal spray or as nasal drops, include aqueous or oily solutions of the therapeutic agent.

Formulations suitable for vaginal administration may be presented as pessaries, tamports, creams, gels, pastes, foams or spray formulations containing in addition to the therapeutic agent such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered therapeutic agent.

It should be understood that in addition to the ingredients, particularly mentioned above, the formulations of the present invention may include other agents conventional in the art having regard to the type of formulation in question, for example, those suitable for oral administration may include flavoring agents. Many variations of the present invention may suggest themselves to those skilled in the art in light of the above detailed disclosure. All such modifications are within the full intended scope of the appended claims.

The present invention is further illustrated by the following examples, which are not to be construed in any way as imposing limitations upon the scope thereof. It will be clear to one of skill in the art that various other modifications, embodiments, and equivalents thereof exist which do not depart from the spirit of the present invention and/or the scope of the appended claims.

Each patent, patent application and reference noted herein is expressly incorporated herein by reference in its entirety.

### EXAMPLES

### Example 1

Preparation of Biotinylated HS

Heparin sulfate (HS) was biotinylated using EZ-Link NHS-LC-Biotin (Pierce). One-half milliliter HS solution (2 mg/ml in NaHCO₃, pH 8.5) was mixed with 0.05 ml of a freshly prepared solution of EZ-Link NHS-LC-Biotin in dimethyl sulfoxide. The mixture was incubated at room temperature for 1 hour. Unconjugated biotin was removed by centrifugation (10,000 RPM) through a Microcon-3 filter (Millipore) followed by dilution with phosphate buffered saline (PBS). This procedure was repeated five times to ensure complete removal of free biotin. Unwanted aldehydes in the reaction were then quenched by incubation with 1 ml of Tris-glycine buffer (25 mM-183 mM, pH 8.3) at room temperature for 20 minutes. The mixture was subjected to three rounds of microfiltration as described above. Biotinylated HS (5 mg/ml in PBS) was aliquoted and stored at −20° C.

### Example 2

Assay of Heparanase Activity

Biotin-labeled HS was digested with heparanase. The reaction mixture containing undegraded and degraded HS was incubated with a biotin-binding plate. Streptavidin-conjugated enzyme was added to the plates, and the reaction was measured by observing the color, indicating the presence of available biotin molecules. A decrease in color reflected HS digestion by heparanase.

Heparanase was diluted in Reaction Buffer (3.33 mM calcium acetate pH 7.0, containing 0.1 mg/ml BSA) to a working concentration (0.01 micro-units to 1 milliunit) Biotin-HS was diluted to a desired concentration in Reaction Buffer. To determine heparanase activity, 10 μl of heparanase solution was mixed with 200 μl of the biotin-HS substrate in a 96 well plate. The reaction was incubated at 43° C. for 1 hour. One hundred microliters of the reaction mixture was added to a hydrated biotin-binding plate and incubated at 37° C. for 30 minutes. Wells were washed five times with buffer and incubated with 100 μl of 1:3000 diluted Streptavidin-enzyme conjugate for 30 minutes at 37° C. The wells were washed five times with assay buffer and incubated for 20 minutes with 100 μl of Substrate solution. Color development in the wells was assessed by measuring optical density at 450 nm in a microplate reader. One unit of enzyme activity was defined as the amount required to generate 1 μmole of hexuronic acid per minute.

### Example 3

Induction and Measurement of Endothelial Heparanase Activity:

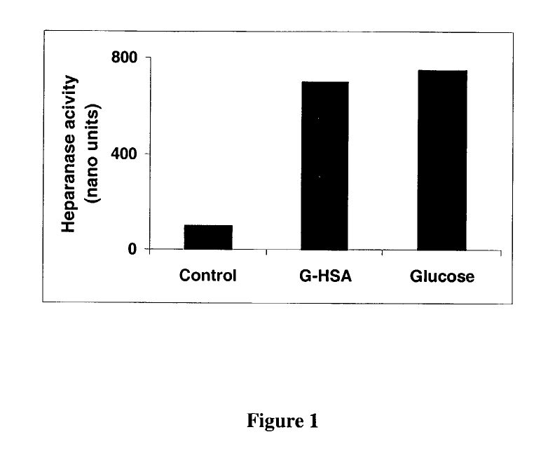

Experiments were done on human microvascular endothelial cells (HMVEC) grown in 48-well plates (˜90% confluency). To induce heparanase activity, culture media in each well was replaced with 200 μl Dulbecco's modified Eagle's medium (DMEM) complemented with 1% bovine serum albumin (BSA) and 100 ng biotinylated HS with or without stimulants (300 μg/ml glycated albumin, 10 ng/ml vascular endothelial growth factor, or 25 mM glucose). Cells were incubated in a cell culture incubator for 16-18 hours and the entire 200 μl media was added to a streptavidin-coated plate and followed by standard color development assay as described in Example 2. To minimize the effects of possible inactivation of heparanase, substrate (biotinylated HS, using the methods of Example 1) was added during the incubation, thus a decrease in the amount of undigested HS represents HS degrading heparanase activity. The decrease in biotinylated HS was correlated with heparanase activity. The amount of undigested HS (which was reduced by different treatments) was then converted to heparanase activity units as shown in FIG. 1.

Treatment of cells with high glucose and glycated albumin resulted in the secretion of approximately 0.7 to 1.5 micro units of heparanase. Unstimulated cells did not secrete any significant amount of heparanase into medium. These data show that agents involved in vasculopathy induce heparanase in endothelial cells.

### Example 4

Immunohistochemistry of Heparanase Expression in Tissues

Heparan sulfate plays a key role in kidney function, and heparanase expression is induced by diabetes-inducing and atherogenic molecules as shown in the Examples presented herein. Induction of heparanase expression was tested in tissues of mice which are used as a model for kidney disease. Mice which are deficient in leptin receptor (db/db mice), show a phenotype that is very similar to patients with type 2 diabetes mellitus. These mice are a useful model in which to study the pathogenesis and treatment of diabetic nephropathy. Mice deficient in apolipoprotein E (apoE) develop atherosclerosis and also develop kidney dysfunction. Heparanase expression in these two mouse models was compared with that of wild type mice.

Kidneys from 2 month old C57BL/6 mice, ApoE-null mice, type II diabetic db/db mice, and ApoB mice were used. Immunohistochemistry was performed on tissue sections fixed in 10% neutral buffered formalin and embedded in paraffin. Sections of 4 μm were deparaffinized and rehydrated, then quenched with endogenous peroxidase in 3% H₂O₂/methanol for 30 minutes. Sections were first incubated with 0.1% BSA/PBS for 20 minutes at room temperature, then incubated with polyclonal rabbit anti-heparanase antibody (1:100 diluted in saline) at 37° C. for 1 hour, then 4° C. overnight, followed by incubation with horseradish peroxidase-conjugated goat anti-rabbit IgG antibody (Sigma Chemical Co.) at 37° C. for 1 hour. Each of these steps was followed by three washes with phosphate buffered saline. Color was developed by using diaminobenzidine as substrate, and positive staining was defined as dark-brown staining. For negative controls, the primary antibody was replaced with 0.1% BSA.

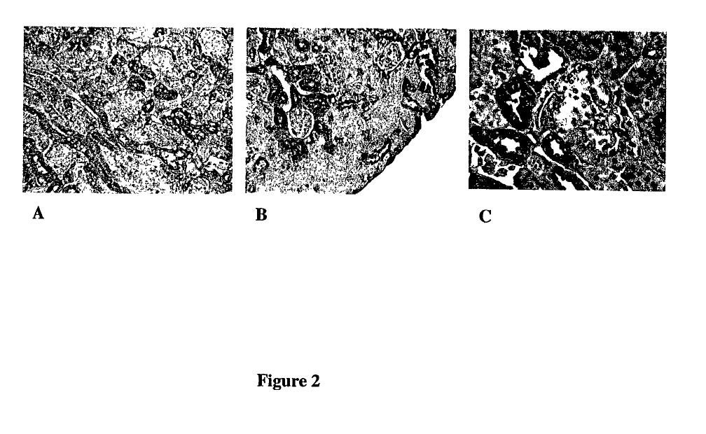

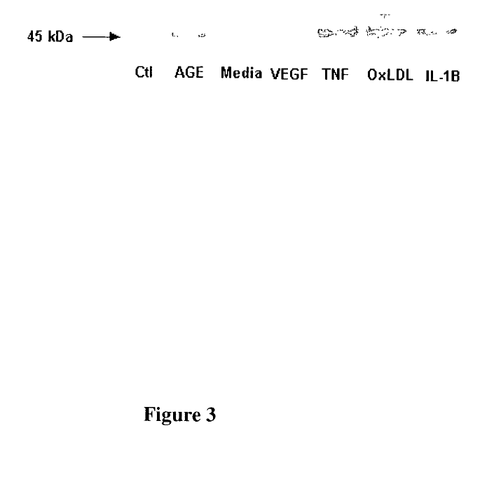

Strong and abundant heparanase staining was observed in kidneys of apoE null mice (FIG. 2B), localized mainly to renal proximal tubular epithelial cells. Staining could also be detected albeit less intensely in some mesangial areas. Heparanase expression in the wild type mouse kidney, however, was significantly less in comparison to that of the ApoE null mouse (FIG. 2A). Like the result with the apoE null mouse, heparanase positive staining was also seen in the db/db mouse kidney relatively more intensely in the renal proximal tubular epithelial cells (FIG. 2C). These data show that heparanase is preferentially induced in kidney dysfunction.

### Example 5

Induction and Measurement of Endothelial Heparanase Protein:

TNF-α has been implicated in the pathophysiology in a number of acute disease states and can contribute to cell death, apoptosis, and organ dysfunction.

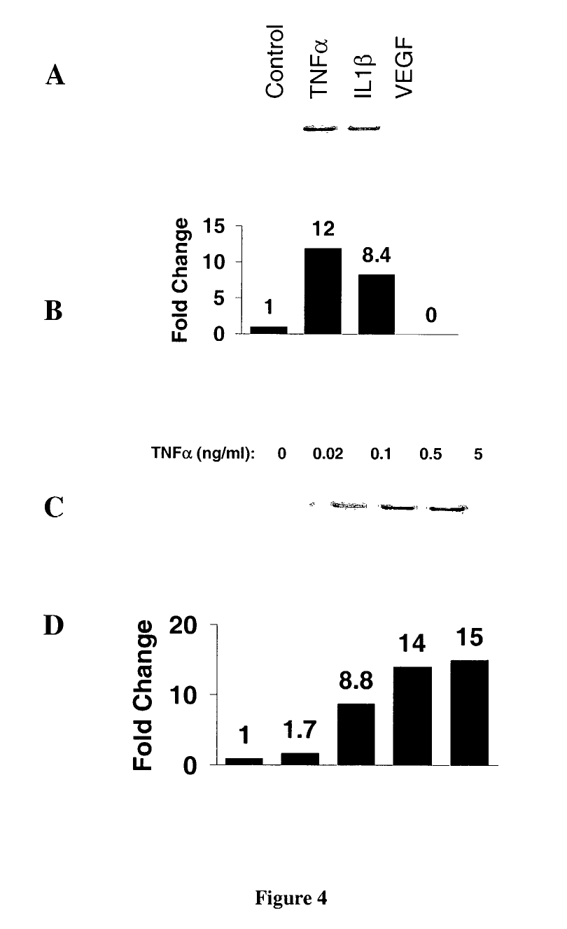

Experiments were done on human microvascular endothelial cells (HMVEC) grown in 48-well plates (˜90% confluency). To induce heparanase activity, culture media was replaced with 200 μl Dulbecco's modified Eagle's medium (DMEM) complemented with 1% bovine serum albumin (BSA) and 100 ng biotinylated HS with or without stimulants (5 ng/ml TGFα, 1 ng/ml IL 1β, or 200 ng/ml VEGF). Cells were incubated in a cell culture incubator for 16-18 hours and the entire 200 μmedia was added to a streptavidin-coated plate and followed by standard color development assay as described in Example 2. The secreted proteins were analyzed by SDS-PAGE and heparanase protein was detected by immunoblotting using polyclonal anti-human heparanase antibody. The results are displayed in FIG. 4A The changes in heparanase expression were also determined.

### Example 6

Dose Dependent Changes in Heparanase Secretion.

Human microvascular endothelial cells (HMVEC) were grown in 48-well plates (˜90% confluency). To induce heparanase activity, culture media was replaced with 200 μl Dulbecco's modified Eagle's medium (DMEM) complemented with 1% bovine serum albumin (BSA) and 100 ng biotinylated HS with 0, 0.02, 1.5 and 5 ng/ml of TNFα. The secreted heparanase was detected by immunoblotting the culture media with anti-human heparanase antibody. The results are shown in FIG. 4C. The changes of heparanase expression determined by densitometric analysis are indicated in FIG. 4D.

### Example 7

Role of Down-Stream Signaling Kinase Pathways in TNFα-Induced Heparanase Secretion by Endothelial Cells:

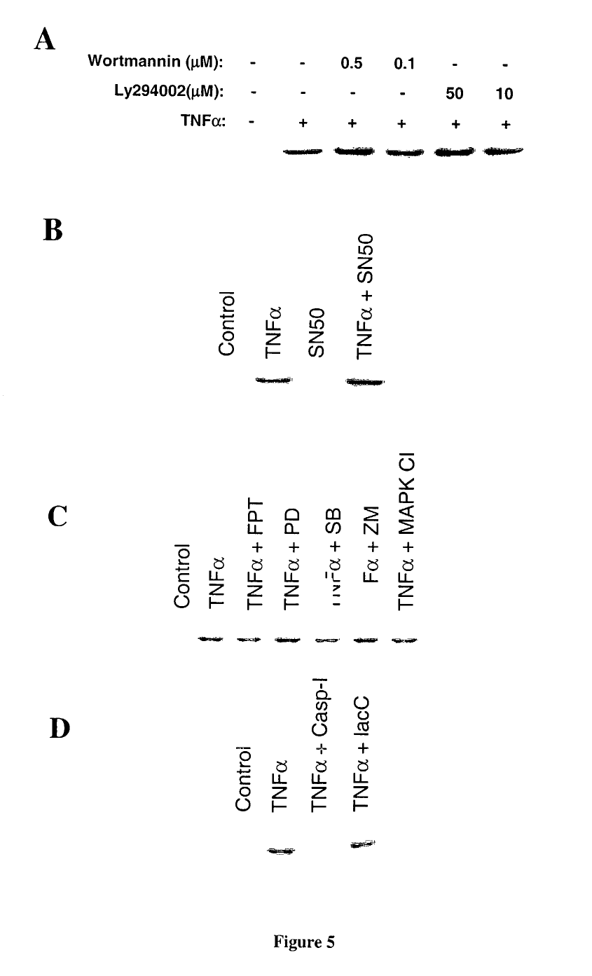

Human microvascular endothelial cells (HMVEC) were grown in 48-well plates (˜90% confluency). To induce heparanase activity, culture media was replaced with 200 μl Dulbecco's modified Eagle's medium (DMEM) complemented with 1% bovine serum albumin (BSA), 100 ng biotinylated HS and TNFα with or without (FIG. 5A) P13 kinase inhibitors (wortmannin, 0.1 μM and 0.5 μM, or Ly294002, 10 μM and 50 μM), (FIG. 5B) NFκB inhibitor (SN50, 10 μM), several MAP kinase inhibitors (FIG. 5C)—either 100 μM FPT inhibitor III (FPT, inhibits Ras processing in cells), 5 μM MAP kinase (MEK), inhibitor PD98059 (PD), 650 nM p38 kinase inhibitor SB203580 (SB), 200 nM c-Raf inhibitor ZM336372 (ZM); or an inhibitor cocktail (Cl) that contains all of the four inhibitors; or broad-spectrum caspase inhibitor III (Casp-I, 10 μM) (FIG. 5D). The cells were incubated with TNFα and the inhibitors for 16 h. The culture media was collected and the secreted proteins were analyzed by SDS-PAGE and heparanase protein was detected by immunoblotting using polyclonal anti-human heparanase antibody. The results are displayed in western blots in FIG. 5. Inhibitors of P13 kinase (A), NFκB (B) or MAP kinases (C) do not inhibit TNFα-induced heparanase whreas caspase inhibition blocked heparanase secretion (D).

### Example 8

Heparanase Expression in Atherosclerotic Regions

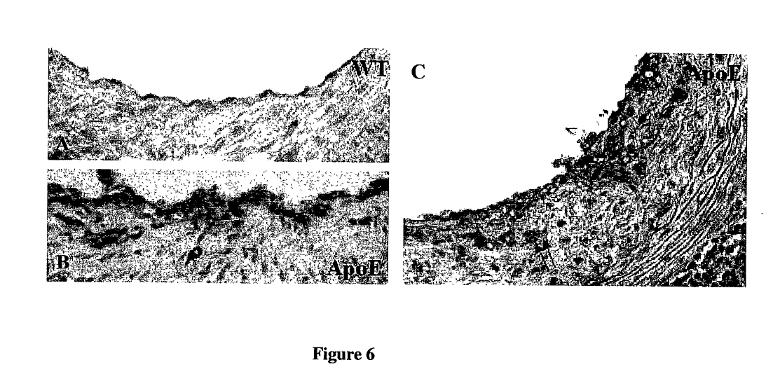

Aortic tissue sections from 3-month old C57BL/6 wild type (WT) (FIG. 6A), 3-month old apoE-null (FIG. 6B) or 1-year old apoE-null mice (FIG. 6C) were immunostained for heparanase expression. Aortic tissues were obtained and fixed in 10% neutral buffered formalin, embedded in paraffin, and 5 μm sections were prepared for immunohistochemistry. After deparaffinization in xylene and rehydration, sections were treated for antigen retrieval in citrate buffer (0.01M, pH 6.0) for 3 minutes in a microwave oven. Endogenous peroxidase activity was quenched with 1.5% H₂O₂/methanol, then tissues were blocked with 5% normal goat serum to eliminate nonspecific background immunostaining.

Sections were incubated with heparanase antibody (1:140 dilution in 1% BSA/PBS) at 37° C. for 1 hr and at 4° C. overnight. After washing with PBS, sections were treated with biotinylated anti-rabbit IgG, followed by avidin-biotin peroxidase complex (Vector laboratories) at room temperature for 1 hr. Color was developed using aminoethy carbozole (AEC) as substrate for 10 minutes. Sections were counterstained with hematoxylin (Zymed). For negative control, primary antibody was replaced by normal rabbit IgG. Heparanase was prominently found in endothelial cells of apoE-null mouse but not wild type mouse. Positive staining can also be seen in some subendothelial matrix, but not in smooth muscle cells. In advanced lesions, strong staining for heparanase was found in both endothelial cells and macrophages of the neointima, see FIG. 6C, indicated by arrows.

### REFERENCES

1. Chantrel F, Moulin B, Hannedouche T. Blood pressure, diabetes and diabetic nephropathy. Diabetes Metab. July 2000;26 Suppl 4:37-44.

2. Rossing P. Promotion, prediction and prevention of progression of nephropathy in type 1 diabetes mellitus. Diabet Med. November 1998;15(11):900-19.

3. Borch-Johnsen K, Anderson, P K., Deckert, T. The effect of proteinuria on relative mortality in type I diabetes mellitus. Diabetologia 28:590-596, 1985

4. Borch-Johnsen K, Feldt-Rasmussen B, Strandgaard S, Schroll M, Jensen J S. Urinary albumin excretion. An independent predictor of ischemic heart disease. Arterioscler Thromb Vasc Biol. August 1999;19(8):1992-7.

5. Deckert T, Feldt-Rasmussen B, Borch-Johnsen K, Kofoed-Enevoldsen A: Albuminuria reflects widespread vascular damage: The Steno hypothesis. Diabetologia 32: 219 -226, 1989

6. Feldt-Rasmussen B. Microalbuminuria, endothelial dysfunction and cardiovascular risk. Diabetes Metab. July 2000;26 Suppl 4:64-6.

7. Makino H, Ikeda S, Haramoto T, Ota Z. Heparan sulfate proteoglycans are lost in patients with diabetic nephropathy. Nephron. 1992;61(4):415-21.

8. Kofoed-Enevoldsen A. Heparan sulphate in the pathogenesis of diabetic nephropathy. Diabetes Metab Rev. July 1995;11(2):137-60.

9. Jensen, T. (1997) Pathogenesis of diabetic vascular disease: evidence for the role of reduced heparan sulfate proteoglycan. Diabetes. 46 (Suppl 2), S98-100

10. Murata K, Yokoyama Y. Acidic glycosaminoglycans in human atherosclerotic cerebral arterial tissues. Atherosclerosis. July 1989;78(1):69-79.

11. Kjellen, L. and Lindahl, U. (1991) Proteoglycans: structure and interactions. Ann. Rev. Biochem. 60, 443-475

12. Rosenberg, R. D., Shworak N. W., Liu J. Schwartz J. J., and Zhang L. (1997) Heparan sulfate proteoglycans of the cardiovascular system. J. Clin. Invest. 99:2062-70

13. Castellot J J Jr, Hoover R L, Harper P A, Karnovsky M J. Heparin and glomerular epithelial cell-secreted heparin-like species inhibit mesangial-cell proliferation. Am J Pathol. September 1985; 120(3):427-35.

14. Tamsma J T, van den Born J, Bruijn J A, Assmann K J, Weening J J, Berden J H, Wieslander J, Schrama E, Hermans J, Veerkamp J H, et al. Expression of glomerular extracellular matrix components in human diabetic nephropathy: decrease of heparan sulphate in the glomerular basement membrane. Diabetologia. March 1994;37(3):313-20.

15. Hollman, J., A. Schmidt, D. von Bassewitz and E. Buddecke. (1989) Relationship of sulfated glycosaminoglycans and cholesterol content in normal and atherosclerotic human aorta. Arteiosclerosis, 9,154-158

16. Volker, W., A. Schmidt, W. Oortmann, T. Broszey, V. Faber, and E. Buddecke. 1990. Mapping of proteoglycans an atherosclerotic lesions. Eur. Heart J. 11:29-40

17. Nakajima, M., Irimura, T. and Nicolson, G. L. (1988) Heparanases and tumor metastasis. J. Cell. Biochem. 36, 157-167

18. Vlodavsky, I, Friedmann, Y., Elkin, M., Aingorn, H., Atzmon, R., Ishai-Michaeli, R., Bitan, M., Pappo, O., Peretz, T., Michal, I., Spector, L., Pecker, I. (1999) Mammalian heparanase: gene cloning, expression and function in tumor progression and metastasis. Nat. Med. 5, 793-802

19. Graham, L. D., Underwood, P. A. (1996) Comparison of the heparanase enzymes from mouse melanoma cells, mouse macrophages, and human platelets. Biochem. Mol. Biol. Int. 39, 563-71.

20. Pillarisetti, S., Obunike, J. C. and Goldberg, I. J. (1995) Lysolecithin induced alterations of subendothelial heparan sulfate proteoglycans increases monocyte binding to matrix J. Biol. Chem. 270, 29760-29765

21. Pillarisetti, S., S. Paka, J. Obunike, L. Berglund, I. Goldberg. (1997) Subendothelial retention of lipoprotein (a). Evidence that reduced heparan sulfate promotes lipoprotein (a) retention by subendothelial matrix. J. Clin. Invest. 100, 867-874

22. Chen S, Cohen M P, Ziyadeh F N. Amadori-glycated albumin in diabetic nephropathy: pathophysiologic connections. Kidney Int. September 2000;58 Suppl 77:S40-4.

23. Friedman E A. Advanced glycation end-products in diabetic nephropathy. Nephrol Dial Transplant. 1999; 14 Suppl 3:1-9.

24. Cohen M P, Masson N, Hud E, Ziyadeh F, Han D C, Clements RS. Inhibiting albumin glycation ameliorates diabetic nephropathy in the db/db mouse. Exp Nephrol. May-June 2000;8(3): 135-43.

25. Cohen M P, Ziyadeh F N. Role of Amadori-modified nonenzymatically glycated serum proteins in the pathogenesis of diabetic nephropathy.J Am Soc Nephrol. February 1996;7(2):183-90. Review.

26. Aiello L P, Wong J S. Role of vascular endothelial growth factor in diabetic vascular complications. Kidney Int. September 2000;58 Suppl 77:S113-9.

27. Cha D R, Kim N H, Yoon J W, Jo S K, Cho W Y, Kim H K, Won N H. Role of vascular endothelial growth factor in diabetic nephropathy. Kidney Int. September 2000;58 Suppl 77:S104-12.

28. Ozaki H, Seo M S, Ozaki K, Yamada H, Yamada E, Okamoto N, Hofmann F, Wood J M, Campochiaro P A. Blockade of vascular endothelial cell growth factor receptor signaling is sufficient to completely prevent retinal neovascularization. Am J Pathol. February 2000;156(2):697-707.

29. Kobayashi K, Forte T M, Taniguchi S, Ishida B Y, Oka K, Chan L. The db/db mouse, a model for diabetic dyslipidemia: molecular characterization and effects of Western diet feeding. Metabolism. January 2000;49(1):22-31.

30. Zhang S H, Reddick R L, Piedrahita J A, Maeda N. Spontaneous hypercholesterolemia and arterial lesions in mice lacking apolipoprotein E. Science. Oct. 16, 1992;258(5081):468-71.

31. Plump A S, Smith J D, Hayek T, Aalto-Setala K, Walsh A, Verstuyft J G, Rubin E M, Breslow J L. Severe hypercholesterolemia and atherosclerosis in apolipoprotein E-deficient mice created by homologous recombination in ES cells. Cell. 1992 Oct 16;71(2):343-53.

32. Chen, G., Paka, L., Kako, Y. and Pillarisetti, S. Apolipoprotein E regulation of mesangial cell growth and kidney function. Circulation 2000 (abstract)

33. Ruoslahti E, Engvall E. Integrins and vascular extracellular matrix assembly. J Clin Invest. Dec. 1, 1997;100(11 Suppl):S53-6.

34. Engel J. Domains in proteins and proteoglycans of the extracellular matrix with functions in assembly and cellular activities. Int J Biol Macromol. June 1991;13(3):147-51. Review

35. Steinberg D. Low density lipoprotein oxidation and its pathobiological significance. J Biol Chem. Aug. 22, 1997;272(34):20963-6

36. Steinberg D, Parthasarathy S, Carew T E, Khoo J C, Witztum J L. Beyond cholesterol. Modifications of low-density lipoprotein that increase its atherogenicity. N Engl J Med. Apr. 6, 1989;320(14):915-24.

37. Chen C H, Jiang W, Via D P, Luo S, Li T R, Lee Y T, Henry P D. Oxidized low-density lipoproteins inhibit endothelial cell proliferation by suppressing basic fibroblast growth factor expression. Circulation. Jan. 18, 2000;101(2):171-7.

38. Ramasamy S, Lipke D W, Boissonneault G A, Guo H, Hennig B. Oxidized lipid-mediated alterations in proteoglycan metabolism in cultured pulmonary endothelial cells. Atherosclerosis. February 1996; 120(1-2): 199-208.

39. Steinbrecher U P, Parthasarathy S, Leake D S, Witztum J L, Steinberg D. Modification of low density lipoprotein by endothelial cells involves lipid peroxidation and degradation of low density lipoprotein phospholipids. Proc Natl Acad Sci USA. June 1984;81(12):3883-7.

40. Murugesan G, Fox P L. Role of lysophosphatidylcholine in the inhibition of endothelial cell motility by oxidized low density lipoprotein. J Clin Invest. Jun. 15, 1996;97(12):2736-44.

41. Rohrbach D H, Hassell J R, Kleinman H K, Martin G R. Alterations in the basement membrane (heparan sulfate) proteoglycan in diabetic mice. Diabetes. February 1982;31(2):185-8.

42. Van den Born J, van den Heuvel L P W J, Bakker M A H, Veerkamp J H, Assman K J M, Weening J J, Berden J H M: Distribution of GBM heparan sulfate proteoglycan core protein and side chains in human glomerular diseases. Kidney Int 43: 454-463, 1993.

43. Tracey K J, Cerami A. Tumor necrosis factor: a pleiotropic cytokine and therapeutic target. Annu Rev Med. 1994;45:491-503.

44. Ferrari R. The role of TNF in cardiovascular disease. Pharmacol Res. 1999 40(2):97-105.

45. Moller D E. Potential Role of TNF-alpha in the Pathogenesis of Insulin Resistance and Type 2 Diabetes. Trends Endocrinol Metab. 2000 11(6):212-217.

46. Hotamisligil G S, Spiegelman B M. Tumor necrosis factor alpha: a key component of the obesity-diabetes link. Diabetes. November 1994;43(11): 1271-8.

47. Nilsson J, Jovinge S, Niemann A, Reneland R, Lithell H. Relation between plasma tumor necrosis factor-alpha and insulin sensitivity in elderly men with non-insulin-dependent diabetes mellitus. Arterioscler Thromb Vasc Biol. 1998 18(8):1199-202.

48. Williams R O, Marinova-Mutafchieva L, Feldmann M, Maini R N. Evaluation of TNF-alpha and IL-1 blockade in collageninduced arthritis and comparison with combined anti-TNF-alpha/anti-CD4 therapy. J Immunol. 2000 165(12):7240-5.

49. Attur M G, Dave M, Cipolletta C, Kang P, Goldring M B, Patel I R, Abramson S B, Amin A R. Reversal of autocrine and paracrine effects of interleukin 1 (IL-1) in human arthritis by type II IL-1 decoy receptor. Potential for pharmacological intervention. J Biol Chem. 2000 275(51):40307-15.

What is claimed is:

1. A method for detecting a change in proteoglycan degrading enzyme activity, comprising,
(a) mixing a sample suspected of containing a proteoglycan degrading enzyme with a composition comprising a first complementary binding partner to form a reaction mixture;
(b) removing an aliquot of the reaction mixture to a second complementary binding partner to bind the first complementary binding partner;
(c) adding a labeled second complementary binding partner;
(d) detecting the label; and
(e) determining the amount of change.

2. The method of claim 1, wherein the proteoglycan degrading enzyme is heparanase.

3. The method of claim 1, wherein the first complementary binding partner is heparin sulfate-biotin.

4. The method of claim 1, wherein the second complementary binding partner is Streptavidin.

5. The method of claim 1, wherein the sample is a bodily fluid or tissue sample.

6. The method of claim 5, wherein the sample is blood, serum, saliva, tissue fluid, urine, tears, plasma, cells, a biopsy section, a tumor, or neoplasm.

7. A method for detecting compounds that inhibit enzyme activity, comprising,
(a) mixing a sample containing a proteoglycan degrading enzyme with a test compound;
(b) adding the mixture of a) with a composition comprising a first complementary binding partner bound to form a reaction mixture;
(c) removing an aliquot of the reaction mixture to a second complementary binding partner to bind the first complementary binding partner;
(d) adding a labeled complementary binding partner;
(e) detecting the label; and
(f) determining the change in amount of proteoglycan.

8. The method of claim 7, wherein the first complementary binding partner is heparin sulfate-biotin.

9. The method of claim 7, wherein the second complementary binding partner is Streptavidin.

10. The method of claim 7 wherein the proteoglycan degrading enzyme has an effect on a proteoglycan substrate containing heparan sulfate.

11. The method of claim 7 wherein the sample comprises fluid from cells that have been pretreated with advanced glycation end-products, TNF-α, OxLDL, IL-1β, or other inflammatory cytokines;

12. A method for treating vasculopathy, comprising administering an effective amount of a therapeutic agent which inhibits proteoglycan degrading enzymes.

13. A method for diagnosing vasculopathy or predicting incipent vasculopathy comprising:
(a) mixing a sample suspected of containing a proteoglycan degrading enzyme with a composition comprising a first complementary binding partner to form a reaction mixture;
(b) removing an aliquot of the reaction mixture to a second complementary binding partner to bind the first complementary binding partner;
(c) adding a labeled second complementary binding partner;
(d) detecting the label; and
(e) determining the amount of change.
(f) comparing the amount of change with a known standard.

14. The method of claim 13, wherein the proteoglycan degrading enzyme is heparanase.

15. The method of claim 13, wherein the first complementary binding partner is heparin sulfate-biotin.

16. The method of claim 13, wherein the second complementary binding partner is Streptavidin.

17. The method of claim 13, wherein the sample is a bodily fluid or tissue sample.

18. The method of claim 13, wherein the sample is blood, serum, saliva, tissue fluid, urine, tears, plasma, cells, a biopsy section, a tumor, or neoplasm.
